(19) United States
(12) Patent Application Publication
(10) Pub. No.: US 2004/0089316 A1
(43) Pub. Date: May 13, 2004

(54) Methods and systems for hair coloring and highlighting

(76) Inventors: Carol Hamilton, New York, NY (US); Michael David, New York, NY (US); Paula Gendel, New York, NY (US); Julia Youssef, Nutley, NJ (US); Patricia Slattery, Brooklyn, NY (US); Kathy Roher, Hartsdale, NY (US); Janice Martell, Flushing, NY (US); Eric Bone, Reuil Malmaison (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/385,302

(22) Filed: Mar. 11, 2003

Related U.S. Application Data
(60) Provisional application No. 60/424,347, filed on Nov. 7, 2002.
(60) Provisional application No. 60/433,789, filed on Dec. 17, 2002.

(51) Int. Cl. A61K 7/13

(52) U.S. Cl. 132/208

(57) ABSTRACT

Various methods, systems and structures relating to hair treatment are disclosed. They include a kit made up of a hair dye and a hair highlight. The kit may contain instructions to apply the highlight while the hair remains moist. A bristled applicator may be included in the kit for use in applying highlight to the hair. Corresponding methods of use and marketing are also disclosed.

This application claims priority to U.S. Provisional Application No. 60/424,347, filed Nov. 7, 2002 and to U.S. Provisional Application No. 60/433,789, filed Dec. 17, 2002, the entire disclosures of both of which are incorporated by reference herein.

### BACKGROUND OF THE INVENTION

1. Field of the Invention

The present invention has application to multi-tonal treatment processes used to color keratinous fibers, such as hair. More particularly, the present invention may relate to a multi-tonal hair treatment process for home use by consumers.

2. Description of Related Art

With age, hair color often dulls and loses multi-tonal effects and natural highlights. That is, color variation typical of youthful hair may be lost or may be muted in the hair of some individuals. Also, regardless of age, often people want to experiment with hair shades different than their natural color. More recently, a variety of hair colors and bleaches may be used in salons to achieve a plurality of effects on the person's hair color. Therefore, a wealth of hair care products exists to vary hair color, and lighten selected strands.

Synthetically, achieving multi-tonal effects typically involves dyeing and highlighting hair. Hair dyeing which typically involves altering the overall color of the hair, usually requires application of a hair dye for a period of time followed by a rinse to remove the dye. During highlighting, the color of groups of selected hair strands may be altered to provide a multi-tonal or contrasting effect. For example, bleach or a lighter dye may be applied to portions or strands of the hair to achieve a lightening of those portions.

Due to its time-consuming nature and multiple steps, such multi-tonal effects are typically provided in the salon. In the salon, strands may be segregated for highlighting before an overall color is applied. This segregation can occur using foil to separate hair to be highlighted from hair to be dyed with an overall color. Alternatively, to avoid damaging the hair, the highlighting of the hair is typically not performed until at least one day after dyeing the hair. The labor intensive nature of multi-tonal effects usually renders a relatively expensive salon treatment.

Home dye treatments may result in a look that is drastic in that the hair becomes substantially a single color all over with very few shade distinctions. Similarly, home highlighting kits may result in a relatively drastic lightening of the hair or may provide unnatural effects.

### SUMMARY OF A FEW ASPECTS OF THE INVENTION

With an exemplary embodiment of a kit and method according to an aspect of the present invention, an improved hair treatment system and process may be obtained that avoid too drastic highlighting. Additionally, the hair treatment system and process may permit the successive use of a dye material followed by a highlighting material. Indeed, according to an aspect of the present invention, the hairs may recover at each use of the kit their original color by the application of the hair dye material before the highlighting material.

It should be understood that the invention could be practiced without having one or more features of any objects, aspects, or embodiments described herein. In addition, such features are exemplary and at least some of them are set forth in the detailed description which follows.

According to an exemplary embodiment, the invention may include a hair care kit that comprises at least one dye compartment comprising a hair dye material and at least one highlight compartment comprising a hair highlight material, and instructions for applying to hair at least some of the dye material the at least one dye compartment and at least some of the highlight material of the at least one highlight compartment so as to achieve multi-tonal highlighting of the hair.

The highlight material may contain a bleaching agent and the hair care kit may be packaged for home use.

The instructions may include directions to apply the bleach to the hair while the hair is moist. In particular, the instructions may include directions to apply dye material to the hair, rinse the dye material from the hair, and then to apply the hair bleach while the hair is still moist from the rinsing.

This kit may also include at least a third, rinse compartment comprising a rinse, and the instructions may include directions for applying the rinse after applying the dye material to the hair. In another aspect, the kit may include at least two third compartments containing two rinses, and the instructions may include directions for applying one of the rinses after the dye material and the other of the rinses after applying the highlight material. The rinses may be applied to rinse the dye material and/or the highlight material, respectively from the hair. At least one of the rinses may include at least one hair conditioner.

According to another aspect of the invention, the kit may comprise an applicator having radially extending bristles. The instructions may comprise directions for applying highlight material via the applicator. The applicator may be sized to hold sufficient highlight material so that a substantially even coating may be applied to hair strands from root to end without the need to reload the applicator.

Yet another aspect of the invention includes a kit wherein the bleach is in powdered form. The kit may also comprise at least two trays and the trays may be configured to nest within each other. The compartments and/or trays of the kit may be labeled in a manner identifying for a user an order of intended use.

According to yet another exemplary embodiment, a hair care kit may comprise at least one dye compartment containing a hair dye material. The hair care kit may further comprise at least one color developing compartment containing an oxidative agent for oxidizing the dye material and developing dye on the hair. The hair care kit also may comprise at least one highlight compartment containing a hair highlight material.

The hair dye material may be chosen from oxidation bases, couplers, and direct dyes, for example. A suitable oxidation base may be chosen from para-phenylenediamines, bisphenylalkylenediamines, para-aminophenols, ortho-aminophenols and heterocyclic bases, and cosmetically acceptable salts of thereof, for example. A suitable coupler may be chosen from meta-phenylenediamines, meta-aminophenols, meta-diphenols, naphthalene couplers and heterocyclic couplers, and the cosmetically acceptable salts of thereof, for example. A suitable direct dye may be chosen from nitro dyes of the benzene series, cationic direct dyes, azo direct dyes, and methine direct dyes, for example

According to another exemplary aspect, the oxidative agent may be selected from peroxide enzymes and oxidase enzymes. For example, the oxidative agent may be chosen from peroxidases, oxidoreductases containing 2 electrons, and oxygenases containing 4 electrons. Also, the oxidative agent may be chosen from uricases and laccases.

In an example, the oxidising agent may be hydrogen peroxide.

The hair highlight material of the hair care kit may be chosen from persalts, such as, for example, persulfate. The oxidative agent may be further configured to be added to the hair light material to enhance the highlight power of the highlight material.

In yet another exemplary embodiment, the hair care kit may comprise at least one highlight enhancing composition which may contain an oxidative agent selected from hydrogen peroxide, for example.

According to another exemplary embodiment, a method of treating keratinous fibers may comprise applying a dye material to the keratinous fibers under conditions sufficient to dye said keratinous fibers and rising the dye material from the keratinous fibers. The method may further comprise applying a highlight material to at least some of the keratinous fibers while the keratinous fibers remain moist from the rinsing. This may effect multi-tonal highlighting of the keratinous fibers. In another aspect, the highlight material, such as bleach, for example, may be applied to the keratinous fibers using an applicator having a portion comprising a core and radially extending bristles.

Applying the highlight material, for example, a bleach, may be performed in a manner so that the moisture in the keratinous fibers at least partially impedes an effect of the bleach, thereby minimizing damage to the keratinous fibers as the result of successive steps of dyeing and bleaching. The method may further comprise towel drying the keratinous fibers after rinsing the dye material from the keratinous fibers.

Yet another exemplary aspect of the invention includes a method of marketing a treatment for keratinous fibers. The method may comprise providing a dye material for the keratinous fibers and a highlight material, for example, a bleach, for the keratinous fibers and associating the dye material and the bleach together. The method may further comprise providing instructions to a user to apply the dye material to the keratinous fibers, to rinse the dye material from the keratinous fibers, and to apply bleach to the keratinous fibers while the keratinous fibers remain moist from the rinsing, so that the moisture impedes an effect of the bleach. In addition, the method may include providing instructions to rinse the bleach from the keratinous fibers in a prescribed time-frame to minimize damage to the keratinous fibers from successive steps of dyeing and bleaching.

The method may further include associating the dye material and the bleach together by packaging the dye material and the bleach together. The method may also comprise associating the dye material and the bleach together by referencing the bleach on a packaging associated with the dye material and/or referencing the dye material on a packaging associated with the bleach.

According to yet another exemplary embodiment, the invention may include a method for treating keratinous fibers comprising applying a dye material to the keratinous fibers, rinsing the dye material from the keratinous fibers, and applying a bleach to the keratinous fibers while the fibers remain moist from rinsing the dye material. The method may further comprise rinsing the bleach from the keratinous fibers not longer than twenty minutes after applying the bleach.

The providing of the instructions may include providing instructions to remove the bleach from the keratinous fibers in a range of 10-25 minutes after applying the bleach.

In yet another exemplary aspect, a method of marketing a treatment process for keratinous fibers comprises providing a consumer with a multistep hair treatment kit, the kit including at least a dye material, a rinse, a bleach, an applicator, and instructions for accessing electronically stored use instructions. The method may further include providing in the electronically stored use instructions directions for using the kit to treat the hair. As an example, the providing the electronically stored use instructions may comprise providing the instructions via at least one of CD-ROM, magnetic storage medium, optical storage medium, flash memory, internet address, and a network address.

The method may further comprise associating the electronically stored use instructions with the treatment process. Associating the electronically stored use instructions may comprise providing a website address on packaging associated with the hair treatment kit, for example. As an alternative, a CD-ROM or other computer readable medium may be packaged or associated with the hair treatment kit.

The method also may comprise providing a timer for tracking progress of the treatment. The timer which may be built into the computer readable medium, may be configured to cue the consumer to end at least one step of the treatment process when a prescribed time limit is reached.

According to yet another aspect, the invention may include a method of marketing a treatment process for keratinous fibers comprising providing the treatment process for the keratinous fibers to at least one host, encouraging the at least one host to hold a gathering of consumers interested in the treatment process and directing the host to teach gathered consumers to use the treatment process by demonstrating the process and having multiple said gathered consumers use the treatment process while at the gathering.

The encouraging may include providing the host an incentive, such as a financial incentive or a product at reduced cost. The method may further comprise enabling the host to sell a treatment process product at the gathering. The method may also include encouraging the host to hold the gathering at the host's residence.

According to yet another exemplary embodiment, a hair care kit for a hair treatment process may comprise at least one dye compartment comprising a hair dye material and at least one highlight compartment comprising a hair highlight material. The kit may further comprise instructions for performing the hair treatment process, the instructions including instructions to apply to hair at least some of the dye material of said at least one dye compartment and at least some of the highlight material of said at least one highlight compartment. The instructions may further comprise instructions for completing the hair treatment process in less than approximately 2 hours. As an alternative, the instructions for completing the hair treatment process may include instructions for completing the process in less than approximately 1.5 hours. In yet another example, the instructions may include instructions for completing the process in about, or less than, 1 hour.

Yet another exemplary embodiment may include a method for treating keratinous fibers which comprises applying a dye material to the keratinous fibers under conditions sufficient to dye the keratinous fibers. The method may further comprise applying a highlight material to at least some of the keratinous fibers after applying the dye material. The method may comprise achieving multi-tonal highlighting of the keratinous fibers. Also, the method may be completed in less than approximately 2 hours. For example, the method may be completed in less than approximately 1.5 hours. Indeed, depending on desired effects, the method may be completed in about, or even less than, 1 hour.

Yet another exemplary embodiment of the invention includes a method of marketing a multistep beauty care process which may comprise airing a television program of at least about 15 minutes in duration. Through the television program, consumers may be educated consumers on how to use a multistep personal care process. Also, during the television program, consumers may be encouraged to request a coupon for purchase of a kit for performing the multistep personal care process. The method may further comprise transmitting the coupon to consumers to incentivize the consumers to purchase the kit at a retail establishment and encouraging the consumers to visit a retail establishment and to redeem the coupon during purchase of the kit at the retail establishment.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG. 1 is an exemplary kit consistent with the present invention.

FIG. 2 is a mascara-type applicator consistent with the present invention.

FIGS. 3a and 3b are examples of instructions, consistent with the present invention.

FIG. 4 is a chart enumerating exemplary aspects of a marketing method consistent with the present invention.

### DESCRIPTION OF THE EMBODIMENTS

Reference will now be made in detail to the present exemplary embodiments of the invention, examples of which are illustrated in the accompanying drawings. Wherever possible, the same reference numbers will be used throughout the drawings to refer to the same or like parts.

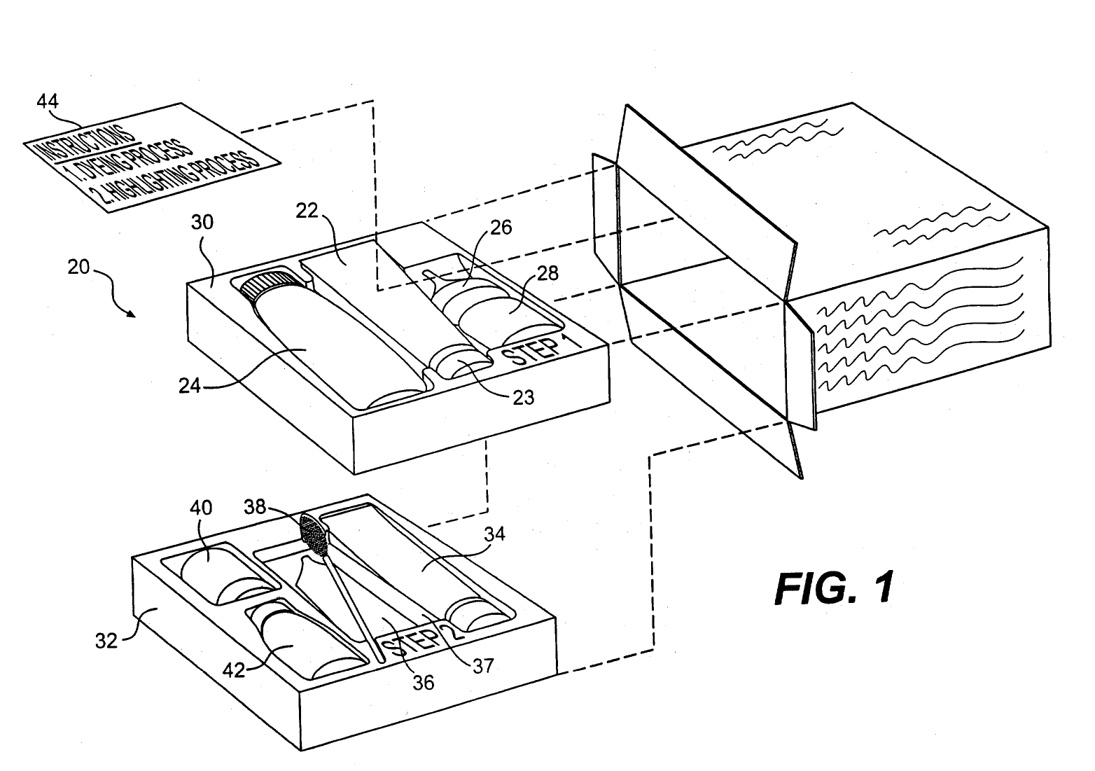

In accordance with the invention there may be provided a hair care kit. As used herein, the term “kit” broadly includes items that are either sold or packaged together. For example, as illustrated in FIG. 1, a kit 20 may include a number of items, described later in greater detail, that are sold together in one box or package. It should be noted that FIG. 1 illustrates an exemplary kit, and is not intended to limit the invention, as claimed. Indeed, the kit of the invention may include more or less items than are illustrated in FIG. 1, and may include varying combinations of items. Similarly, the kit may be packaged in a variety of manners.

While the kit may be distributed to end users through salons, one aspect of the invention involves distributing kits to consumers through retail sales channels such as drugstores, department stores, grocery stores, and cosmetic stores and other retail or user accessible discount or wholesale chains. This aspect of the invention may permit consumers to purchase the product for home use.

In accordance with the invention, the kit may include at least one dye compartment containing a hair dye material. As used herein, the term “compartment” refers to any receptacle, regardless of shape, material, or closure, which serves a containing function. For example, the term “compartment” as used throughout, broadly includes, (but is not limited to) the interior of a tube, sack, canister, can, tub, bottle, jar, packet, carton, box, envelope, or any other vessel. Components of the dye may be contained in a single receptacle, or may be divided amongst multiple receptacles. For example, as illustrated in FIG. 1, the dye material may be contained in multiple compartments, with a color base contained within tube 22 and a color developer within bottle 24.

While the invention, in its broadest sense is not limited to any particular dye formulation or compound, one example of a suitable dye may be a two part formulation, the first part being a multi-tonal translucent gel crème color base containing water, deceth-3, laureth-12, oleth-30, deceth-5, trideceth-2 carboxamide mea, ammonium hydroxide, glycerin, oleic acid, hexadimethrine chloride, oleyl alcohol, polyquaternium-6, fragrance, ethanolamine, ammonium thiolactate, resorcinal, p-Phenylenediamine, ascorbic acid, edta, poly-quaternium-24, hydroxyethyl bis (N-Hydroxyethyl-p-Phenylenediamine), HC1, m-Aminophenol, 2,4-Diaminophenoxyethanol HC1.

The second component of the dye may be, for example, a multi-tonal crème developer containing water, hydrogen peroxide, cetearyl alcohol, trideceth-2-carboxamide mea, ceteareth-30, glycerin, pentasodium pentetate, sodium stannate, tetrasodium pyrophosphate [D0000/0].

According to an exemplary aspect, a translucent gel-crème formula may be used for the dye material, which may have a consistency that combines the spreadability of a gel and the conditioning of a cream. Cationic polymers comprising conditioning agents may be selected for the formula, which may permit deep conditioning and replenishing of the hair. Moreover, the dye formula may faciliate application to the hair and facilitate even coverage. The formula may also facilitate rinsing of the hair, leaving the hair smooth, detangled and ready for the highlight application step. Also, the level of colorants may be balanced for each base color dye, which may improve total gray coverage and may created reflective hair shades.

According to another exemplary embodiment, the dye compartment may comprise an oxidation base conventionally used in oxidation dyeing, for example para-phenylenediamines, bisphenylalkylenediamines, para-aminophenols, ortho-aminophenols, and heterocyclic bases and the cosmetically acceptable salts of these compounds.

Suitable para-phenylenediamines that may be used may be chosen from, for example, para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine and their addition salts with an acid.

Among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and their addition salts with an acid, may be more suitable, for example.

Suitable bisphenylalkylenediamines that may be used may be chosen from, for example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and their addition salts with an acid.

Suitable para-aminophenols that may be used may be chosen from, for example, para-aminophenol, 4-amino-3-methyl phenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and their addition salts with an acid.

Suitable ortho-aminophenols that may be used may be chosen from, by way of example, 2-aminophenol, 2-amino-5-methylphenol, 2-amino-6-methylphenol, 5-acetamido-2-aminophenol, and their addition salts with an acid.

Suitable heterocyclic bases that may be used may be chosen from, for example, pyridine derivatives, pyrimidine derivatives, and pyrazole derivatives.

Suitable pyridine derivatives may be chosen from the compounds described, for example, in Patents GB 1,026,978 and GB 1,153,196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their addition salts with an acid, for example.

Suitable pyrimidine derivatives may be chosen from the compounds described for example in Patents DE 2,359,399; JP 88-169,571; JP 05-163,124; EP 0,770,375 or Patent Application WO 96/15765, such as 2,4,5,6-tetra-aminopyrimidine, 4-hydroxy-2,5,6-triaminopyrimidine, 2-hydroxy-4,5,6triaminopyrimidine, 2,4-dihydroxy-5,6-diaminopyrimidine, 2,5,6-triaminopyrimidine, and the pyrazolopyrimidine derivatives such as those mentioned in Patent Application FR-A-2,750,048 and among which there may be mentioned pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)-(2-hydroxy-ethyl)amino]ethanol, 2-[(7-aminopyrazolo[1,5-a]-pyrimidin-3-yl)-(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine, 3-amino-5-methyl-7-imidazolylpropylaminopyrazolo[1,5-a]pyrimidine, their tautomeric forms, when a tautomeric equilibrium exists and their addition salts with an acid.

Suitable pyrazole derivatives may be chosen from the compounds described in Patents DE 3,843,892, DE 4,133,957 and Patent Applications WO 94/08969, WO 94/08970, FR-A-2,733,749 and DE 195 43 988 such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and their addition salts with an acid.

According to another exemplary embodiment, the dye compartment may further comprise one or more couplers which may be conventionally used for dyeing keratinous fibers. For example, suitable couplers may be chosen from meta-phenylenediamines, meta-aminophenols, meta-diphenols, naphthalene couplers and heterocyclic couplers and the cosmetically acceptable salts of these compounds.

By way of example, 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis-(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis-(β-hydroxyethylamino)toluene and their addition salts, may be used as suitable couplers.

The coupler(s) may be present in a quantity ranging from approximately 0.001% to approximately 10% by weight approximately of the total weight of the dyeing composition, and/or from approximately 0.005% to approximately 6%. The additional oxidation base(s) may be present a quantity ranging from approximately 0.001% to approximately 10% by weight approximately of the total weight of the dyeing composition, and/or from approximately 0.005% to approximately 6%.

According to an exemplary aspect, the salts which can be cosmetically used in the context of the composition of the invention for oxidation bases and the couplers present may be chosen from the addition salts with an acid such as the hydrochlorides, hydrobromides, sulfates, citrates, succinates, tartrates, lactates, tosylates, benzenesulfonates, phosphates and acetates, for example, and the addition salts with a base such as sodium hydroxide, potassium hydroxide, aqueous ammonia, amines or alkanolamines, for example.

In yet another exemplary embodiment, the dye compartment may comprise one or more direct dyes which may be chosen from nitro dyes of the benzene series, cationic direct dyes, azo direct dyes, methine direct dyes, for example.

The appropriate medium for dyeing, also called dye carrier, may include water or a mixture of water and at least one organic solvent to solubilize the compounds which might not be sufficiently soluble in water. By way of organic solvent, suitable examples may be chosen from C₁-C₄ lower alkanols, such as ethanol and isopropanol; for example, polyols and polyol ethers such as 2-butoxyethanol, propylene glycol, propylene glycol monomethyl ether, diethylene glycol monoethyl ether and monomethyl ether, for example, and aromatic alcohols such as benzyl alcohol or phenoxyethanol, for example, and mixtures thereof.

The solvents may be present in proportions ranging from about 1% to about 40% by weight approximately relative to the total weight of the dyeing composition, and/or from about 5% to about 30% by weight approximately.

In yet another exemplary embodiment, the dyeing composition may comprise various adjuvants conventionally used in compositions for dyeing hair, such as, for example, anionic, cationic, nonionic, amphoteric or zitterionic agents, or mixtures thereof, anionic, cationic, nonionic, amphoteric or zitterionic polymers, or mixtures thereof, inorganic or organic thickening agents, and in particular anionic, cationic, nonionic and amphoteric associative polymeric thickeners, antioxidants, penetrating agents, sequestrants, perfumes, buffers, dispersing agents, conditioning agents such as, for example, volatile or nonvolatile, modified or unmodified silicones, film-forming agents, ceramides, preservatives, and opacifying agents.

The above adjuvants may be present in a quantity, for each of them, ranging from about 0.01% to about 20% by weight relative to the weight of the composition, for example.

Of course, persons skilled in the art may choose this or these possible additional compounds such that advantageous properties which may be intrinsically attached to the oxidation dyeing composition in accordance with the invention may not, or may not substantially, be impaired by the addition(s) envisaged.

The pH of the dyeing composition in accordance with an exemplary aspect of the invention may range from about 3 to about 12, and/or from about 5 to about 11, for example. The pH may be adjusted to the desired value by means of acidifying or alkalinizing agents customarily used in dyeing keratinous fibres or alternatively using conventional buffer systems.

Suitable acidifying agents may be chosen, for example, from inorganic or organic acids, such as hydrochloric acid, orthophosphoric acid, sulphuric acid, carboxylic acids, such as acetic acid, tartaric acid, citric acid, lactic acid, for example, and sulphonic acids.

Suitable alkalinizing agents may be chosen, by way of example, from aqueous ammonia, alkali metal carbonates, alkanolamines such as mono-, di- and triethanolamines and derivatives thereof, sodium or potassium hydroxides and the compounds having the following formula (III):

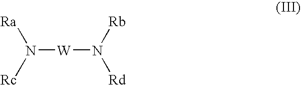

in which W is a propylene residue which is optionally substituted with a hydroxyl group or a C₁-C₄ alkyl radical; Rₐ, R_{b}, R_{c} and R_{d}, which are identical or different, represent a hydrogen atom, a C₁-C₄ alkyl or C₁-C₄ hydroxyalkyl radical.

An exemplary dyeing composition may be provided in various forms, such as in the form of liquids, creams, gels, or any other form appropriate for dyeing keratinous fibers, such as human hair, for example.

Other components also may be packaged in the kit. For example, as illustrated in FIG. 1, the kit may include an applicator cap 26 and a pair of gloves, for example contained within a two-piece glove holder 28 of an “egg” structure. All of components 22, 24, 26, and 28 may be packaged in tray 30. Tray 30, which may contain the components of the hair dyeing process, may nest within another tray 32, which may be configured to hold the contents of a hair highlighting process.

The kit may also include at least one highlight compartment comprising a hair highlight material. As with the hair dye material, the hair highlight material may be completely contained within a single compartment (as defined previously) or may be contained within multiple compartments. For example, as illustrated in FIG. 1, a highlight base may be contained within tube 34 and tonal powder may be contained within a packet 36, to be mixed with the highlighting base before use.

In a broad sense, the hair highlight material may be any ingredient or combination of ingredients for adding a tone to the hair. For example, the highlight material may include a bleaching agent or a component of a bleaching agent. The highlight material also may comprise a decolorizer, for example. While the invention, in its broadest sense, is not limited to any particular highlight formulation or compound, one suitable example includes a two component bleaching formulation, mixed, prior to application, by the user. By way of example, the first component may be an illuminating crème developer comprising water, hydrogen peroxide, cetearyl alcohol, trideceth-2-carboxamide mea, ceteareth-30, glycerin, pentasodium pentetate, sodium stannate, tetrasodium pyrophosphate [D0000/0]. The second component of the highlight formulation may be, for example, a micro-fine illuminating powder containing potassium persulfate, sodium silicate; sodium persulfate; scrylates/c10-30 alkyl acrylate cross polymer, urea, kaolin, magnesium stearate, ammonium chloride, diethylhexyl sodium sulfosuccinate, vp/va copolymer, polydecene, sodium metasilicate, magnesium peroxide edta, titanium dioxide, cyamopsis tetragonoloba (guar) gum, sodium carboxymethyl starch, sodium benzoate [D0000/0]. An exemplary quantity contained within packet 36 may be 10 gr.

Alternatively, the highlight material may be a hair coloring agent, such as a dye. Preferably, the color of the highlight material is selected to complement the base color contained in the kit. Moreover, the highlighting material may be calibrated to provide the appropriate amount of highlighting so as to harmonize with the respective base color packaged in the kit. For example, the highlighting material may reveal and complement the undertones of the color base, thereby creating an interplay of multi-tonal color and light, highs and lows. The highlight material may comprise a thickening agent so as to hinder dripping of the material during application and to allow the material to stay in place once applied to the hair strands.

The kit may further include an applicator, such as a wand 38, an additional pair of gloves contained within an “egg”-type enclosure 40 and a hair-conditioner or rinse contained in bottle 42.

In accordance with the invention, the kit may additionally include instructions for applying to hair at least some of the contents of said at least one dye compartment and at least some of the contents of said at least one highlight compartment. Instructions may take one or more forms. For example, they may be printed on one or more of the containers, 22, 24, 34, 36, or 42, they may be printed on the trays 30 and/or 32, or they may printed on the box 29 in which trays 24 and 32 are packaged. Alternatively, or in addition, instructions may be contained on a separate medium contained within the box. For example, as illustrated in FIG. 1, instructions may be printed on paper stock 44.

The instructions may contain one or more of texts and graphics. Alternatively, or in addition, the instructions may be contained on an electronic medium contained within the box, and/or printed instructions may contain an address to an internet site at which further instructions or demonstration might be observable.

The electronic media might be, for example, a compact disc, DVD, magnetic disc, flash memory, or any other mechanism for storing information for display.

While the instructions are discussed later in greater detail, they may contain directions for applying highlight to the hair subsequent to the application of a hair dye so as to obtain a multi-tonal highlighting of the hair. For example, the instructions may, depending on the particulars of the kit, describe application of the highlight material using the enclosed wand 38. The instructions may direct the user to apply the highlight material to the hair before the hair is completely dry. For example, the instructions might direct application of the highlight material after towel drying. It is commonly known that towel drying leaves the hair moist, and in accordance with the invention, remaining moisture in the hair may have a beneficial protective effect during the highlighting process. The instructions may also direct the user to complete the entire hair treatment process in less than approximately 2 hours, or in less than approximately 1.5 hours, or in less than approximately 1 hour. As an example, the instructions may direct the user to complete the entire hair treatment process in about 1 hour. Further, the instructions regarding the length of time for the completion of the entire process may depend on the manufacturer's intended effects and/or the consumer's desired effects on the hair.

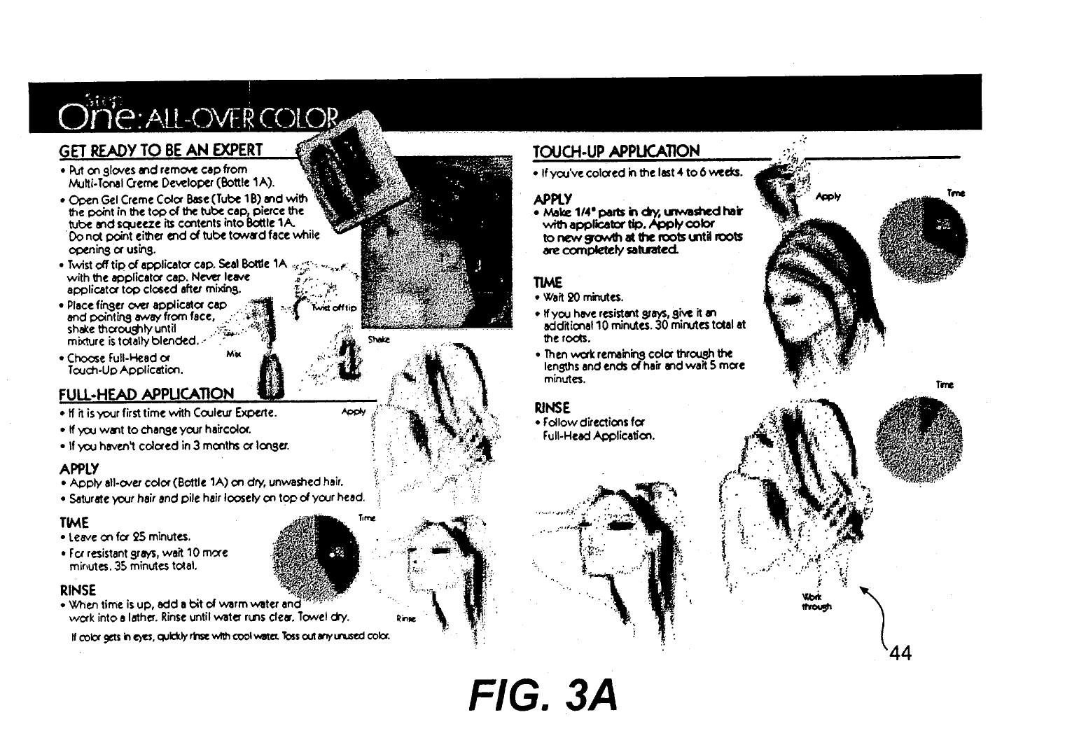

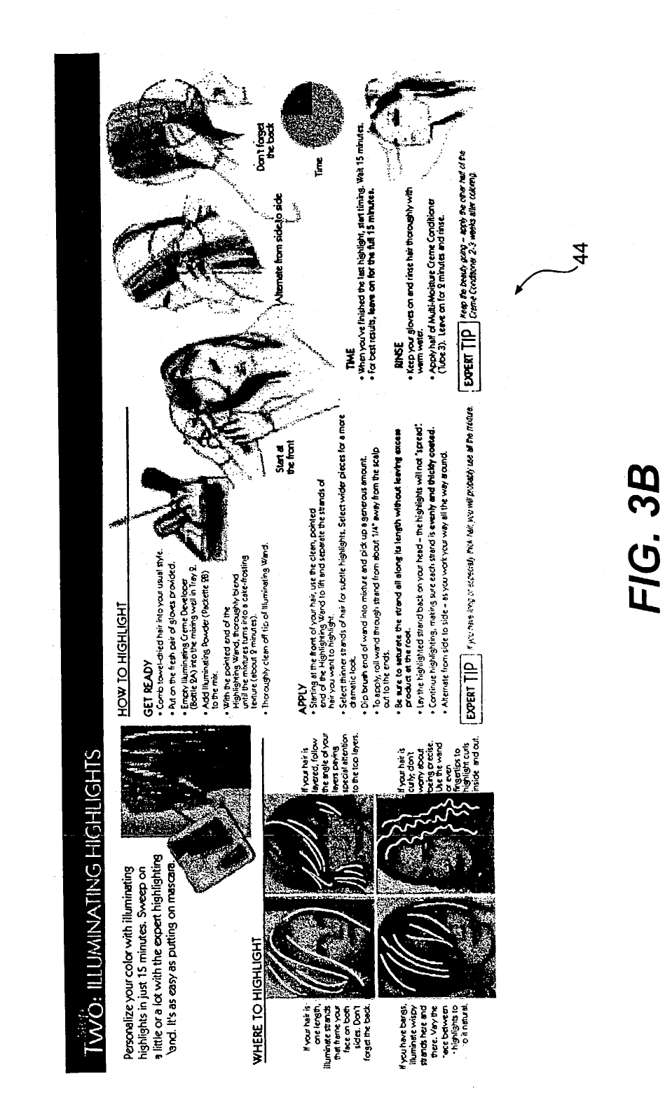

The instructions may vary, depending upon the details of the kit and the manufacturer's intended use. Exemplary instructions are discussed later in connection with methods of the invention. By way of example only, one embodiment exemplary of instructions 44 is depicted in FIG. 1, and another exemplary embodiment of instructions 48 is depicted in FIG. 3.

As previously discussed, the kit 20 may include applicator cap 26. Applicator cap 26 may be threaded in a manner similar to cap 23 of color tube 22. Thus, when cap 23 is removed from color tube 22, applicator cap 26 may be secured to tube 22 in its place. Also as discussed, enclosure 28 may include a pair of gloves. The gloves may be made of latex, other forms of plastic, or any suitable material for protecting the user's hands from the materials contained within compartments 22, 24, 34, 36, and 42. It may be advantageous to use disposable gloves enabling the user to immediately dispense with them following use. Gloves contained within enclosure 40 may have an identical construction to the gloves contained within enclosure 28. While it may be preferable to include two pairs of gloves, one pair for each of the dyeing and highlighting processes, the kit may be configured with a single pair of gloves, or no gloves at all.

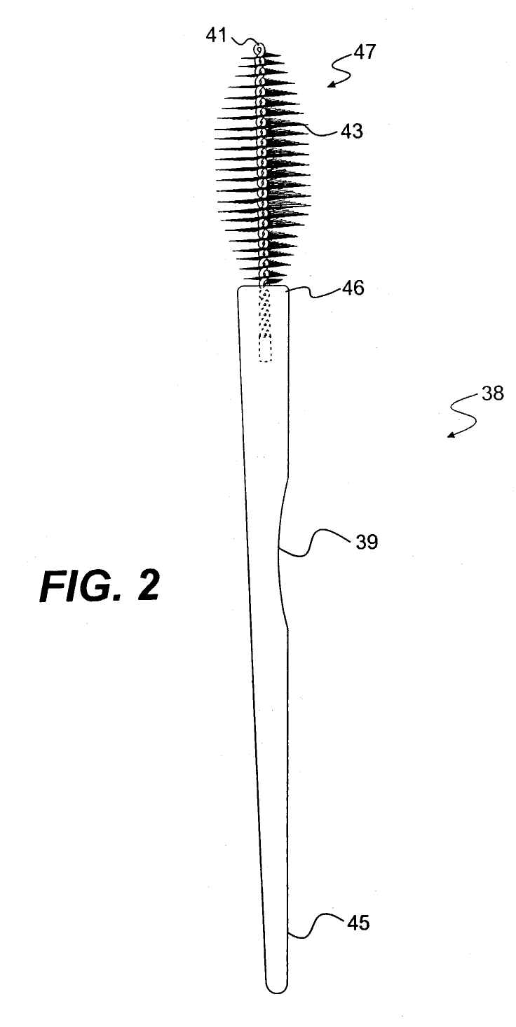

As previously discussed, the kit may also include an applicator, such as a wand 38, schematically illustrated in greater detail in FIG. 2. As an example, the wand 38 may be a mascara-type wand. While the applicator, in its broadest sense, is not limited to any particular configuration, one suitable configuration employs an elongated handle 39 with imbedded wire(s) 41, which may be made of stainless steel, for example. The wire may be twisted to secure bristles 43 to a head end 46 of the applicator. The construction may involve two separate wire twisted together or a single bent wire twisted upon itself. Alternatively, other materials to which bristles may be attached may be used for the core of the bristled head. By way of example, the bristled portion 47 of the applicator may assume the shape of a tapered oval (football) with approximately seventeen rows of bristles at 0.400 break. A suitable length for the bristled portion may be one and one-half inches from the free end of the bristled portion to the end proximate the handle 39. The bristles may be made of nylon. The head defined by the bristle ends may taper from a dimension of about 0.5″ at a wide central region, to about 0.2″ at opposite ends of the bristled head 47. The bristled head portion also may have other suitable shapes, such as, for example, a cylindrical shape, a tapered shaped from the handle toward the free end of the bristled portion, etc.

The bristles, which may have ends that radially extend from a core defined by twisted wire 41, may be made of any material capable of holding and applying highlight material. The twisting may be right to left or left to right, such as is disclosed in U.S. Pat. No. 6,345,923, entitled Make-Up Brush and Method for Manufacturing Such a Brush, filed Jan. 5, 2001, and issued Feb. 12, 2002, the entire disclosure of which is incorporated herein by reference.

Rinse container 42 may hold a hair wash, a hair conditioner or a combination of both. While the invention, in its broadest sense, is not limited to any particular rinse formulation, a suitable exemplary formulation may include water, cetearyl alcohol, gylcerin, behentrimonium chloride, euphorbia cerifera (candelilla) wax, amodimethicone, cetyl esters, isopropyl alcohol, fragrance, methylparaben, trideceth-12, prunus armeniaca (apricot) kernel oil, chlorhexidine dihydrochloride, cetrimonium chloride [D0000/0]. Although a single rinse container is illustrated in the figures, one or more additional rinse materials may be included in the kit.

The inclusion of two trays 24 and 32, may serve multiple functions. First, they may help segregate the components of the kit into those components that are used for the dyeing process and those that are used for the highlighting process. One or more of the indentations in the trays may serve a dual function. For example, they may be shaped to conform to the contours of a container to hold the container in place. And when the container is removed, one or more indentations may be used as a reservoir in which to mix a hair treatment formulation. Alternatively, the tray may contain a reservoir that does not conform to the contour of a container, but which serves a function of a mixing or holding reservoir. For packaging, one of the trays may nest within the other to reduce package size and/or component shifting during shipping. The trays may also be positioned in an external packaging box in a face to face manner. Also, one or both of the trays may be provided with a cover (not shown), for example a vacuum form cover. While the trays do not need to be mutually exclusive, in an exemplary embodiment, one of the trays may hold substantially all the components of the dyeing process while the other holds substantially all the components of the highlighting process.

To avoid misuse and facilitate user friendliness, the trays may be labeled in order to facilitate proper use, and therefore may serve an instructional function as well as an organization function. For example, the hair dyeing tray may contain markings to indicate that it should be used first and the hair highlighting tray may be marked to indicate that its contents should be used after dyeing. For example, the markings may be as simple as “Step 1” on the dyeing tray and “Step 2” on the highlighting tray. Moreover, only those items needed for a particular step may be held in the tray corresponding to that step.

Another aspect of the invention may involve a method of treating keratinous fibers comprising applying a dye to the keratinous fibers under conditions sufficient to dye said keratinous fibers. As embodied herein, and by way of example, this may be accomplished using conventional or unconventional hair dyes. Examples of suitable dyes for the method include any of those described above. While the details of dyeing hair may vary depending on the hair dye used, the desired effect, and personal preference, one example may involve donning gloves to protect the hands, and manually applying a fluid, such as a gel base, on dry unwashed hair. The fluid may come prepackaged in usable form or may be packaged as components that require mixing before use. For example, as illustrated in FIG. 1, a gel cream base contained in tube 22 may be squeezed through applicator cap 26 into a bottle of color developer 24. Applicator cap 26 may then be transferred to bottle 24 and the bottle shaken with a finger over the hole in the applicator cap 26 to prevent spillage during mixing. Thereafter, the mixed contents in bottle 24 may be dispensed by squeezing the contents through the applicator cap 26 onto dry unwashed hair to saturate the hair. The hair may then be piled loosely on the top of the user's head and left for twenty-five minutes. For resistant grays, for example, the user may be instructed to wait an additional ten minutes. For touch-up applications, the dye might be applied to only new growth at the roots. To this end, a user may be instructed to make one-quarter inch parts in dry unwashed hair using the tip of applicator cap 26. Bottle 24 can then be squeezed to saturate only the roots, which may require a waiting time of twenty minutes. For resistant grays, an additional ten minutes may be required. Thereafter, the remaining color may be worked through the length and the ends of the hair followed by an additional five minutes of waiting time. Skilled artisans would understand that the time periods mentioned above for leaving the dye on the hair are exemplary and other time periods may also be used and may vary according to the particular effects desired, such as, for example, intensity of color, etc.

After the dye has been applied and the waiting time elapsed, the invention may include rinsing the dye from the keratinous fibers. This may be accomplished by applying warm water to the head, working into lather, and then rinsing until the water runs clear. Thereafter, excess moisture may be removed from the hair through, for example, towel drying. In an exemplary embodiment, the dye may be rinsed using a conditioning rinse.

The invention may further include applying a highlight material to the keratinous fibers while the keratinous fibers remain moist from rinsing to thereby effect highlighting of said keratinous fibers. In accordance with the invention, the highlighting material may include a formulation that has a bleaching and/or decolorizing effect. Alternatively, or additionally, the formulation may have a coloring effect. For example, if during the dyeing stage, the hair is colored to a first shade of red, the highlight may be an alternate shade of red or a different color entirely.

While some embodiments of the invention may not require application of highlight to moist hair, in one embodiment, highlight application to moist hair may be beneficial, particularly when the highlight is a bleach. Moisture in the hair may reduce absorption of bleach, providing a desired effect and minimizing hair damage. As used herein, the term “moist” may include a range of moisture from wet to partially dry, such as through towel drying. It is generally understood that while towel drying removes excess moisture, it is not sufficient to remove all moisture. Therefore, the term “moist” may refer to hair that has been towel dried.

To apply the highlight material, the user might first don a fresh pair of gloves such as those contained within enclosure 40 on tray 32. The contents of bottle 34 may then be emptied into mixing well 37 of tray 32. An illuminating powder from packet 36 may then be added to the mix. Thereafter, the tip end 45 of handle 39 of the wand 38 may be used to blend the mixture until the mixture turns into a cake-frosting texture. This may take up to approximately two minutes. Thereafter, the tip end 45 of the handle 39 may be cleaned.

With the highlighting material mixed in the mixing well 37, the user might again employ the tip end 45 of handle 39 to lift and separate strands of hair for highlighting, a strand of hair typically consisting of multiple hairs. By selecting thinner strands, i.e., relatively less hairs, subtle highlights may be achieved. By selecting wider strands, i.e., relatively more hairs, a more dramatic look may be achieved. After strands are selected, the user may be instructed to dip the brush end 47 of applicator 38 into the mixing well 37. The brush end (i.e., the bristled portion) may be sized sufficiently to pick up an amount of highlight capable of covering and entire strand from root to end. While such a large brush is preferable, the invention, in its broadest sense, is not limited to any particularly sized brush. Exemplary aspects of an applicator are discussed later in greater detail.

To apply the highlight to the selected strand of hair, the user may be instructed to roll bristled head 47 of the applicator 38 through the strand from about a quarter inch away from scalp out to the ends, saturating the strand along its length without leaving excess product at the root. Thereafter, the highlighted strand may be laid back on the user's head and the process repeated with another strand, making sure that each strand is evenly and thickly coated. For best results, the user may be instructed to alternate from side to side as user works her way all around the head. Upon completion of the highlighting process, the user may leave the highlight in the hair for about fifteen minutes. Of course, the time may vary depending on desired results and the composition of the highlighting material.

Following highlighting, and while the user still dons the gloves, the user may be instructed to rinse the hair thoroughly with warm water and apply at least some of the conditioner contained within container 42. The user may further be instructed to leave the conditioner in the hair for a period of time, such as two minutes, before rinsing. In addition, the user may be instructed to retain some of the conditioner for use at a later time, such as 2-3 weeks after coloring.

As discussed previously, the highlight may be a bleach, a component of a bleach, a bleaching agent, or a component of a bleaching agent, for example. By applying the bleach material to moist hair, the moisture may at least partially impede an effect of the bleach, thereby minimizing damage to the hair as a result of successive steps of dyeing and bleaching.

After dyeing and highlighting, most if not all of the components of the kit may be discarded. Particularly, wand 38 and the gloves contained within enclosures 28 and 40 may be discarded. Likewise, trays 30 and 32 may be disposed of together with each of the various containers. If conditioner is retained within container 42, as discussed previously, container 42 may be saved for a later application.

One method consistent with an exemplary embodiment of the invention involves treating keratinous fibers by applying dye material to the keratinous fibers under conditions sufficient to dye the keratinous fibers. This may be accomplished, as discussed earlier, by applying a hair dye material to the hair. Thereafter, the dye material may be rinsed from the keratinous fibers. This may be accomplished using water, a prepackaged rinse material, or a combination of water or a prepackaged rinse material. The rinse material may contain a shampoo and/or a conditioner.

Following rinsing, and while the keratinous fibers remain moist from the rinsing, the invention may involve applying a highlight material to at least some of the keratinous fibers to thereby effect highlighting of keratinous fibers. In accordance with one aspect of the invention, and as discussed earlier, the highlight material may be applied using a bristled mascara-type wand. However, in its broader sense, the highlight material may be applied using any application technique. In other words, some aspects of the invention do not require the use of any particular applicator.

As previously discussed, towel drying of the hair followed by a successive bleaching before the hair fully dries, may sufficiently impede an effect of the bleach to avoid undue hair damage. Alternatively, other levels of moisture may be effective as well. For example, the hair may be wrung out by hand, or squeegeed with the edge of the hand to remove excess moisture. Alternatively, a period of drip-drying or air drying may work equally well so long as moisture from the dyeing/rinsing step remains in the hair at the time of bleaching.

A multi-tonal highlighting hair treatment process according to an exemplary aspect of the invention and which may include, for example, at least the steps of applying a dye to the hair followed by applying a bleach to the hair, may be completed from start to finish in less than approximately 2 hours. For example, the process may be completed in less than approximately 1.5 hours and/or 1 hour. Also, according to an exemplary aspect, the process may be completed in about 1 hour. The overall time period for completing the multi-tonal hair color process, and also the relative time periods for completing the steps of dyeing the hair and highlighting the hair, may vary according to the desired effects on the hair, such as, for example, the intensity of the overall dye color and/or the intensity of the contrasting, for example, highlighted, portions of the hair.

When an applicator is used to apply the highlight material, it may be preferable to employ an applicator that holds sufficient highlight material to evenly coat a strand of hair without the need to reload the applicator with the highlight material. This ensures more even application, which may result in a more pleasing aesthetic quality. For example, the wand 38 may be configured to hold enough highlight material in the bristled portion 47 to cover a strand of hair from a location near the root to substantially the distal end of the hair strand, regardless of the length of the user's hair. Thus, since the product is intended to be sold over-the-counter to consumers, it may be preferable to design the applicator with a holding capacity sufficient to cover both long and short strands of hair. Such holding capacities may be expressed in a number of ways.

For example, the surface area of the bristles 43 may be consistent with that of the wand 38 described in connection with FIG. 2, which may be calculated from dimensions provided. Acceptable ranges may be plus or minus 50%, 40%, 30%, 20%, 10% and 5%. In addition, the highlight holding capacity of the applicator may be consistent with that of the wand 38 described in connection with FIG. 2, which may be calculated from dimensions provided. Acceptable ranges may be plus or minus 50%, 40%, 30%, 20%, 10% and 5%).

As another example for determining the holding capacity of an exemplary embodiment of the applicator, the bristled portion may have the capacity to hold greater than approximately 1.2 grams of the highlighting material used in the hair highlighting process, such as for example, the highlighting material formed from a mixture of an illuminating crème developer comprising water, hydrogen peroxide, cetearyl alcohol, trideceth-2-carboxamide mea, ceteareth-30, glycerin, pentasodium pentetate, sodium stannate, tetrasodium pyrophosphate [D0000/0] and a micro-fine illuminating powder comprising potassium persulfate, sodium silicate; sodium persulfate; scrylates/c10-30 alkyl acrylate cross polymer, urea, kaolin, magnesium stearate, ammonium chloride, diethylhexyl sodium sulfosuccinate, vp/va copolymer, polydecene, sodium metasilicate, magnesium peroxide edta, titanium dioxide, cyamopsis tetragonoloba (guar) gum, sodium carboxymethyl starch, sodium benzoate [D0000/0]. Components of one such mixture are currently sold by L'Oreal under the trade name Couleur Experte™. In an exemplary embodiment, the bristled portion may have the capacity to hold from approximately 1.2 grams to approximately 3 grams of the above-mentioned highlight material mixture. In yet another exemplary embodiment, the bristled portion may have a capacity to hold about 2 grams of the above-mentioned highlight material mixture.

According to another exemplary aspect, the wand 38 may have a bristled head portion configured to fit within an envelope, for example, an imaginary cylinder, defined by the overall length of the bristled portion and by a radius measured from the core to a bristle end farthest from the core. Such an envelope (e.g., the smallest cylindrical envelope that bounds the bristled head portion) may define a volume greater than approximately 1500 mm³. In an exemplary embodiment, the volume of such an envelope may range from about 3000 mm³ to about 6000 mm³, for example the volume may range from about 4300 mm³ to about 5500 mm³.

The bristle length of the applicator, when the applicator is of the mascara type, may be longer than conventional mascara bristles. For example they may range from about 2 to 3 inches or more, down to conventional mascara bristle lengths. An acceptable length is about one-half an inch at the widest point of bristled head 47, when the bristled head 47 is approximately one and one-half inches long. Other acceptable ranges may approximately fall within two of the following dimensions, measured in inches, each combination of the following list being intended to be within the scope of the invention: 5/16, ⅜, 7/16, 9/16, ⅝, 11/16, ¾, 13/16, ⅞, 1, 1 1/16, 1⅛, 1 3/16, 1¼, 1 15/16, 1⅜, 1 7/16, 1 9/16, 1⅝, 11/16, 1¾, 1 13/16, 1⅞, 2 1/16, 2⅛, 2 3/16, 2¼, 2 15/16, 2⅜, 2 7/16, 2 9/16, 2⅝, 2 1/16, 2¾, 2 13/16, 2⅞, and 3.

Of course, the invention in its broadest sense, is not limited to any particular bristle dimension, and does not even require an applicator, let alone one that is bristled. Even when the invention employs a bristled applicator, bristle lengths other than those listed above are intended to fall within the broad scope of the invention.

According to another aspect of the invention, the invention may simply involve the application of a bleach or a bleaching agent to the hair using an applicator having a portion comprising a core and radially extending bristles. As mentioned, previously, the invention may include the use of a non-conventionally oversized bristled applicator head to facilitate the bleaching process. Indeed, one aspect of the invention may involve the structure of the applicator itself, as previously described. Another aspect may involve the use of a non-conventionally sized applicator to apply dye or highlight to the hair. As previously mentioned, such a structure permits the hair to be treated from about the root to the end, without the need to “reload” the brush. As a result, more even application may be possible.

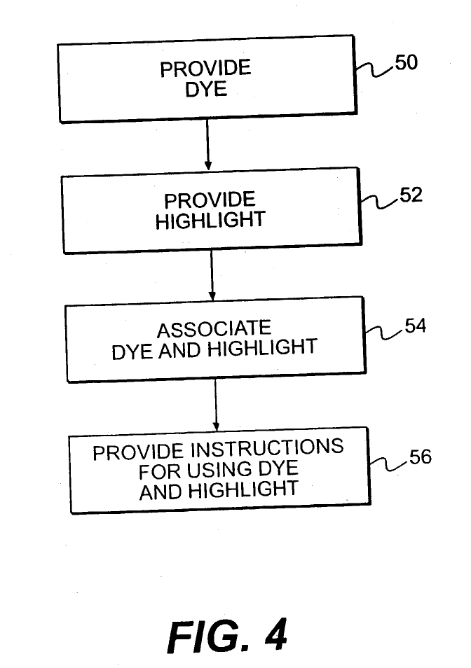

Another aspect of the invention involves a method of marketing a treatment for keratinous fibers comprising providing a dye material for the keratinous fibers and a highlight material for the keratinous fibers and associating the dye material and the highlight material together. As embodied herein, providing a dye material and providing a highlight material (50, 52, respectively in FIG. 4) may include one or more of manufacturing, contracting, purchasing, packaging, or otherwise making available dye and highlight materials. Examples of dye and highlight materials are discussed earlier, and are not repeated in this discussion.

The invention may also include associating the dye material and highlight material together (54 in FIG. 4). As used herein, the term “associating” includes any mechanism for identifying to a user that the dye material and the highlight material compliment each other and may be used in succession. This may include packaging the highlight material and the dye material together in the same packaging materials. Alternatively, the dye and highlight materials may be sold separately but may be marked in a manner suggesting that one be used with the other. Associating 54 may also include advertising the complimentary nature of the dye and the highlight materials, or providing incentives, such as discounts or promotions for purchasing both products. Associating 54 might also include placing on the packaging of one or more of the dye and the highlight, the suggestion to purchase the other of the highlight.

In accordance with the invention, a method may also include providing instructions to a user to apply dye material to the keratinous fibers, to rinse the dye material from the keratinous fibers and to apply the highlight material to the keratinous fibers while the keratinous fibers remain moist from rinsing so that the moisture impedes an effect of the bleach. In accordance with the invention, the provision 56 of instructions may take one of many forms as previously discussed. In addition, the instructions need not explicitly state the moisture impedes an effect of the bleach, but may simply direct the user to follow a procedure that provides that result. For example, the instructions may simply advise the user to apply the highlight after towel drying.

The invention may further including providing instructions to rinse the highlight material from the keratinous fibers in the prescribe time-frame to minimize damage to the keratinous fibers from successive steps of dyeing and highlighting. Again, this provision of instructions need not state the result of minimizing damage, but may merely include directions that leads to that result. For example, the instructions may indicate that the highlight should be rinsed within a period of, for example, 10-25 minutes after completing the application. Of course, the time-frame depends on the details of the formulation and the manufacturer's intended results and/or the user's desired results. For some stronger bleaches, it may be beneficial to instruct the user to rinse the bleach from the fibers not longer than 20 minutes after applying the bleach. For others, 15 minutes may be the instructed time-frame.

Also, the providing step 56 may include providing instructions to complete a multi-tonal hair treatment process, which may include at least the steps of applying of the dye and the bleach in less than approximately 2 hours. For example, from start to finish, the process of dying and bleaching the hair may take less than approximately 1.5 hours. Indeed, depending on desired effect, the entire process may even take about, or less than, approximately 1 hour.

Another aspect of the invention may include a marketing method that includes providing a consumer with a multi-step treatment kit, the kit including at least a dye, a rinse, a bleach, an applicator, and directions for accessing electronically stored use instructions. The electronically stored use instructions may be contained on a computer readable medium within the kit, such as a CD, DVD, flash memory, magnetic storage device, other optical storage medium, or any other storage device for holding information. Alternatively, the directions may include the address of a networked-based set of instructions, such as might be posted on an Internet-based web page.

The invention may also include providing in the electronically stored use instructions directions for using the kit to treat the hair. For example, the instructions set forth in FIGS. 3a and 3b may be provided electronically. The electronic instructions may include graphics or motion picture to help guide the user through the hair treatment process. The electronic instructions may also include a timer configured to cue the consumer to end at least one step of the treatment process when a prescribed time limit is reached.

The invention may also include a marketing method that includes providing a treatment process for keratinous fibers through at least one host. The treatment process may include, for example, the multi-tonal product discussed previously, or may involve any hair treatment process, such as hair coloring, without highlights, hair highlighting without hair coloring, or other applications for the hair. In accordance with this method, the invention may involve encouraging at least one host to hold a gathering of consumers interested in the treatment process. The gathering may be in the form of a party held at the host's home, at a community center, or at some other location capable of holding the invitees. In this way, the market may be opened to individuals who might otherwise be reluctant to experiment with such a product for fear that they may make a mistake or they may not be able to achieve the results that are promised by the product. By gathering a group of individuals together, those concerns may be alleviated. Thus, in accordance with the invention the method may include directing a host to teach gathered consumers to use the treatment process by demonstrating the process. In addition, the invention may include encouraging gathered consumers to use the treatment process while at the gathering. In this way, if the consumers are able to observe first hand an experienced user demonstrate the product and perhaps assist them in using the product for the first time, the consumers might be less reluctant to use the product.

The host may be provided with a financial incentive to hold such product gatherings. The incentive may include remuneration for each person who either attends or purchases a product. In addition, the attendees may receive coupons for purchase of product in the future, and each time such a coupon is redeemed, the host may receive a residual payment. Alternatively, the user might be provided with product at reduced cost and may be able to sell the product to others and earn a profit. The product may be made available at the gathering so that invitees may either purchase product for use at the gathering or purchase product to take home and use at another time. Preferably, the gatherings are held at the user's residence to make the process less intimidating and more inviting.

Another exemplary embodiment of the invention may include a method of marketing a multi-step personal treatment product, for example, a hair treatment product. The method may comprise airing a television program, for example, an infomercial, that is at least approximately 15 minutes in duration. Through the television program consumers may be educated on how to perform a multi-step personal care process. For example, directions for performing a hair treatment process, such as the multi-tonal hair treatment process described above, may be provided. For example, the entire hair treatment process, or representative portions thereof, may be actually performed on a person's hair during the television program. Also, during the infomercial, consumers may be provided information about a retail establishment the consumer may visit to purchase products needed to perform the hair treatment process. Also, during the infomercial, consumers may be provided an incentive to visit a retail establishment and purchase the products needed to perform the hair treatment process. Such incentive may be in the form of a coupon for one or more of the products needed for the hair treatment process or may be in the form of a discounted or free item related to (or unrelated to) the products needed for the hair treatment product. Other types of incentives also may be provided an would be obvious to those having ordinary skill in the art. In an exemplary embodiment, the method may comprise encouraging, during the television program, consumers to request a coupon for purchase of a kit for performing the multistep personal care process, for example. To this end the consumer may be encouraged to place a phone call or visit a Web Site in order to request a coupon. The method may also comprise transmitting the coupon to consumers to incentivize the consumers to purchase the kit at a retail establishment; and encouraging the consumers to visit a retail establishment and to redeem the coupon during purchase of the kit at the retail establishment.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The following claims are hereby incorporated into this Description of Embodiments, with each claim standing on its own as a separate preferred embodiment of the invention.

What is claimed is:

1. A hair care kit, comprising:
at least one dye compartment comprising a hair dye material;
at least one highlight compartment comprising a hair highlight material; and
instructions for applying to hair at least some of the hair dye material of said at least one dye compartment and at least some of the hair highlight material of said at least one highlight compartment so as to achieve multi-tonal highlighting of the hair.

2. The method of claim 1, wherein the hair highlight material comprises a bleaching agent.

3. The hair care kit of claim 2, further comprising an applicator comprising bristles radially extending from an applicator core, wherein the instructions further comprise directions to use the applicator in a manner that causes bleach to be applied to the radially extending bristles, and thereafter to contact at least portions of the hair with the bristles.

4. The hair care kit of claim 2, wherein the instructions further comprise directions for applying the hair bleach subsequent to applying the dye material.

5. The hair care kit of claim 4, wherein the instructions further comprise directions for rinsing the hair after applying the dye material, and applying the bleach to the hair while the hair remains moist from rinsing.

6. The hair care kit of claim 4, wherein the instructions further comprise directions for rinsing the hair after applying the dye material, towel drying the hair after rinsing, and applying the bleach to the hair after towel drying.

7. The hair care kit of claim 3, wherein the instructions further comprise directions for applying the bleach to the hair while the hair remains moist.

8. The hair care kit of claim 1, further comprising at least one rinse compartment containing a rinse, said at least one rinse compartment differing from the at least one dye compartment and the at least one highlight compartment.

9. The hair care kit of claim 8, further comprising at least two rinse compartments.

10. The hair care product of claim 9, wherein contents of at least one of the rinse compartments contains a hair conditioner.

11. The hair care product of claim 10, wherein more than one rinse compartment contains a hair conditioner.

12. The hair care kit of claim 1, further comprising at least one rinse compartment containing a hair rinse, and an applicator having radially extending bristles.

13. The hair care kit of claim 12, wherein more than one of the rinse compartments contain a hair rinse, wherein the highlight is a hair bleach, and wherein the hair bleach is provided in powder form.

14. The hair care kit of claim 1, further comprising at least one rinse compartment containing a hair rinse, and wherein the instructions further comprise directions for rinsing the hair between dyeing and highlighting.

15. The hair care kit of claim 1, further comprising at least two trays, one of the trays for holding at least some dyeing materials and the other tray for holding at least some highlighting materials.

16. The hair care kit of claim 15, wherein said two trays are configured to nest within each other.

17. The hair care kit of claim 1, wherein said dye compartment and said highlight compartment are labeled in a manner identifying for a user an order of intended use.

18. The hair care kit of claim 1, wherein the instructions further comprise directions to use the applicator in a manner that causes highlight to be applied to the radially extending bristles, and thereafter to contact at least portions of the hair with the bristles.

19. The hair care kit of claim 1, wherein the instructions further comprise directions for rinsing the hair after applying the dye material, and applying the highlight material to the hair while the hair remains moist from rinsing.

20. The hair care kit of claim 1, wherein the instructions further comprise directions for rinsing the hair after applying the dye material, towel drying the hair after rinsing, and applying the highlight material to the hair after towel drying.

21. The hair care kit of claim 1, wherein at least one of the trays includes a reservoir therein, and wherein the instructions direct a user to deposit at least one of the dye material and the highlighting material into the reservoir.

22. The hair care kit of claim 21, wherein the reservoir is a depression in one of the trays.

23. The hair care kit of claim 1, wherein the instructions further comprise instructions for completing the applying of at least some of the dye material of the at least one dye compartment and at least some of the highlight material of the at least one highlighting compartment to the hair so as to achieve the multi-tonal highlighting effects in under about 1 hour.

24. A method of treating keratinous fibers comprising:
applying a dye material to the keratinous fibers under conditions sufficient to dye said keratinous fibers;
rinsing the dye material from the keratinous fibers; and
applying a highlight material to at least some of the keratinous fibers while the keratinous fibers remain moist from the rinsing, to thereby effect multi-tonal highlighting of said keratinous fibers.

25. The method of claim 24, wherein applying a highlight material includes applying a bleach.

26. The method of claim 25, wherein applying the bleach is performed in a manner so that the moisture in the keratinous fibers at least partially impedes an effect of the bleach, thereby minimizing damage to the keratinous fibers as the result of successive steps of dyeing and bleaching.

27. The method of claim 24, wherein the keratinous fibers include hair.

28. The method of claim 24, further comprising towel drying the keratinous fibers after the rinsing of the dye from the keratinous fibers.

29. The method of claim 24, wherein applying the highlighting material includes the use of an applicator having bristles radiating from a central core.

30. The method of claim 24, wherein the applying of the dye to the keratinous fibers comprises applying simultaneously or sequentially the dye material and an oxidative agent for oxidising the dye.

31. A method of treating keratinous fibers comprising:
applying a dye material to the keratinous fibers;
rinsing the dye material from the keratinous fibers;
applying bleach to the keratinous fibers using an applicator having a portion comprising a core and radially extending bristles, the applying occurring while the keratinous fibers remain moist from the rinsing.

32. The method of claim 31, wherein the keratinous fibers comprise hair.

33. The method of claim 31, wherein the core is substantially straight.

34. The method of claim 31, wherein a combined surface area of the bristles is capable of holding an amount of bleach sufficient so that regardless of hair length a user can coat a hair strand from a location near hair roots to a location near hair ends without having to load the applicator with bleach more than once.

35. The method of claim 31, wherein the bristles of the applicator are capable of holding greater than about 1.2 grams of a highlight material mixture.

36. The method of claim 35, wherein the highlighting material mixture comprises
an illuminating crème developer comprising water, hydrogen peroxide, cetearyl alcohol, trideceth-2-carboxamide mea, ceteareth-30, glycerin, pentasodium pentetate, sodium stannate, tetrasodium pyrophosphate [D0000/0], and
a micro-fine illuminating powder containing potassium persulfate, sodium silicate; sodium persulfate; scrylates/c10-30 alkyl acrylate cross polymer, urea, kaolin, magnesium stearate, ammonium chloride, diethylhexyl sodium sulfosuccinate, vp/va copolymer, polydecene, sodium metasilicate, magnesium peroxide edta, titanium dioxide, cyamopsis tetragonoloba (guar) gum, sodium carboxymethyl starch, sodium benzoate [D0000/0].

37. The method of claim 35, wherein the bristles of the applicator are capable of holding from about 1.2 grams to about 3 grams of the highlight material mixture.

38. The method of claim 31, further comprising disposing of the applicator after applying the bleach.

39. The method of claim 31, wherein a smallest cylindrical envelope bounding the bristles of the portion of the applicator defines a volume greater than approximately 1500 mm³.

40. The method of claim 39, wherein the cylindrical envelope defines a volume ranging from about 3000 mm³ to about 6000 mm³.

41. The method of claim 39, wherein the smallest cylindrical envelope defines a volume ranging from about 4300 mm³ to about 5500 mm³.

42. A method of treating keratinous fibers comprising:
applying a dye material to the keratinous fibers;
rinsing the dye material from the keratinous fibers;
applying highlight material to the keratinous fibers using an applicator having a portion comprising a core and radially extending bristles, the applying occurring while the keratinous fibers remain moist from the rinsing.

43. The method of claim 42, wherein the bristles of the applicator are capable of holding greater than about 1.2 grams of a highlight material mixture.

44. The method of claim 43, wherein the highlighting material mixture comprises
an illuminating crème developer comprising water, hydrogen peroxide, cetearyl alcohol, trideceth-2-carboxamide mea, ceteareth-30, glycerin, pentasodium pentetate, sodium stannate, tetrasodium pyrophosphate [D0000/0], and
a micro-fine illuminating powder containing potassium persulfate, sodium silicate; sodium persulfate; scrylates/c10-30 alkyl acrylate cross polymer, urea, kaolin, magnesium stearate, ammonium chloride, diethylhexyl sodium sulfosuccinate, vp/va copolymer, polydecene, sodium metasilicate, magnesium peroxide edta, titanium dioxide, cyamopsis tetragonoloba (guar) gum, sodium carboxymethyl starch, sodium benzoate [D0000/0].

45. The method of claim 43, wherein the bristles of the applicator are capable of holding from about 1.2 grams to about 3 grams of the highlight material mixture.

46. The method of claim 42, wherein a smallest cylindrical envelope bounding the bristles of the portion of the applicator defines a volume greater than approximately 1500 mm³.

47. The method of claim 46, wherein the smallest cylindrical envelope defines a volume ranging from about 3000 mm³ to about 6000 mm³.

48. The method of claim 46, wherein the smallest cylindrical envelope defines a volume ranging from about 4300 mm³ to about 5500 mm³.

49. A method of marketing a treatment for keratinous fibers, the method comprising:
providing a dye material for the keratinous fibers and a bleach for the keratinous fibers;
associating the dye material and the bleach together;
providing instructions to a user to apply the dye material to the keratinous fibers, to rinse the dye material from the keratinous fibers, and to apply bleach to the keratinous fibers while the keratinous fibers remain moist from the rinsing, so that the moisture impedes an effect of the bleach; and
providing instructions to rinse the bleach from the keratinous fibers in a prescribed time-frame to minimize damage to the keratinous fibers from successive steps of dyeing and bleaching.

50. The method of claim 49, wherein the keratinous fibers comprise hair.

51. The method of claim 49, wherein the associating of the dye material and the bleach together includes packaging the dye material and the bleach together.

52. The method of claim 49, wherein the associating of the dye material and the bleach together includes at least one of referencing the bleach on a packaging associated with the dye material and referencing the dye material on a packaging associated with the bleach.

53. The method of claim 49, wherein associating the dye material and the bleach together includes packaging the dye material and the bleach together.

54. A method of treating keratinous fibers comprising:
applying a dye material to the keratinous fibers;
rinsing the dye material from the keratinous fibers;
applying a bleach to the keratinous fibers while the keratinous fibers remain moist from rinsing the dye material; and
rinsing the bleach from the keratinous fibers not longer than twenty minutes after applying the bleach.

55. The method of claim 54, wherein the rinsing of the bleach from the keratinous fibers comprises rinsing the bleach approximately fifteen minutes after applying the bleach.

56. The method of claim 54, wherein the keratinous fibers comprise hair.

57. A method of marketing a treatment for keratinous fibers, the method comprising:
providing a dye material for the keratinous fibers and a bleach for the keratinous fibers; and
providing instructions to users to apply the dye material to the keratinous fibers, to rinse the dye material from the keratinous fibers, to apply the bleach to the dyed keratinous fibers shortly after rinsing the dye material, and to remove the bleach from the keratinous fibers at a time period of no longer than 20 minutes after applying the bleach.

58. The method of claim 57, wherein the providing of the instructions includes providing instructions to remove the bleach from the keratinous fibers at approximately 15 minutes after applying the bleach.

59. The method of claim 57, wherein the keratinous fibers comprise hair.

60. An applicator configured to apply highlight material to hair, the applicator comprising:
an elongated handle;
a brush head having a series of bristles connected to the handle, the bristles being configured to hold sufficient highlight material, so that when the applicator bristles are loaded with highlight material, an entire hair strand may be coated without reloading the applicator with highlight material.

61. The applicator of claim 60, wherein the bristles are approximately one-half an inch in length at a widest location of the brush head.

62. The applicator of claim 61, wherein the bristles are between 0.3 inches and 3 inches at a widest location of the brush head.

63. A method of applying a hair treatment, the method comprising:
selecting a strand of hairs from the head of person undergoing application;
dipping a bristled portion of a brush in a reservoir of fluid to load the brush with fluid, the brush being configured to hold sufficient fluid to coat substantially the entire strand of the hair from a location proximate the roots of the hair to a location proximate the ends of the hair.

64. The method of claim 63, wherein the bristled portion is capable of holding greater than about 1.2 grams of a highlight material mixture.

65. The method of claim 64, wherein the highlighting material mixture comprises
an illuminating crème developer comprising water, hydrogen peroxide, cetearyl alcohol, trideceth-2-carboxamide mea, ceteareth-30, glycerin, pentasodium pentetate, sodium stannate, tetrasodium pyrophosphate [D0000/0], and
a micro-fine illuminating powder containing potassium persulfate, sodium silicate; sodium persulfate; scrylates/c10-30 alkyl acrylate cross polymer, urea, kaolin, magnesium stearate, ammonium chloride, diethylhexyl sodium sulfosuccinate, vp/va copolymer, polydecene, sodium metasilicate, magnesium peroxide edta, titanium dioxide, cyamopsis tetragonoloba (guar) gum, sodium carboxymethyl starch, sodium benzoate [D0000/0].

66. The method of claim 64, wherein the bristle portion is capable of holding from about 1.2 grams to about 3 grams of the highlight material mixture.

67. The applicator of claim 63, wherein a smallest cylindrical envelope bounding the bristled portion defines a volume greater than approximately 1500 mm³.

68. The method of claim 67, wherein the smallest cylindrical envelope defines a volume ranging from about 3000 mm³ to about 6000 mm³.

69. The method of claim 67, wherein the smallest cylindrical envelope defines a volume ranging from about 4300 mm³ to about 5500 mm³.

70. A method of marketing a treatment process for keratinous fibers, the method comprising:
providing a consumer with a multi-step hair treatment kit, the kit including at least a dye material, a rinse, a bleach, an applicator, and directions for accessing electronically stored use instructions; and
providing in the electronically stored use instructions directions for using the kit to treat the hair.

71. The method of claim 70, wherein providing the electronically stored use instructions comprises providing the instructions via at least one of CD-ROM, magnetic storage medium, optical storage medium, flash memory, internet address, and a network address.

72. The method of claim 70, further comprising associating the electronically stored use instructions with the treatment process.

73. The method of claim 72, wherein the associating of the electronically stored use instructions comprises providing a website address on packaging associated with the hair treatment kit.

74. The method of claim 72, wherein the associating of the electronically stored use instructions comprises providing a CD-ROM within packaging associated with the hair treatment kit.

75. The method of claim 70, wherein the keratinous fibers include hair.

76. The method of claim 70, further comprising a timer for tracking progress of the treatment, the timer being configured to cue the consumer to end at least one step of the treatment process when a prescribed time limit is reached.

77. A method of marketing a treatment process for keratinous fibers, the method comprising:
providing the treatment process for the keratinous fibers to at least one host;
encouraging the at least one host to hold a gathering of consumers interested in the treatment process;
directing the host to teach gathered consumers to use the treatment process by demonstrating the process and having multiple said gathered consumers use the treatment process while at the gathering.

78. The method of claim 77, wherein encouraging includes providing the user with a financial incentive.

79. The method of claim 77, wherein encouraging includes providing the host with a product at reduced cost.

80. The method of claim 77, further comprising enabling the host to sell a treatment process product at the gathering.

81. The method of claim 77, wherein the host is encouraged to hold the gathering at the host's residence.

82. A hair care kit comprising:
at least one dye compartment comprising a hair dye material,
at least one color developing compartment comprising an oxidative agent for oxidizing the dye material and developing dye on the hair,
at least one highlight compartment comprising a hair highlight material.

83. The hair care kit of claim 82, wherein the hair dye material is selected from oxidation bases, couplers, and direct dyes.

84. The hair care kit of claim 83, wherein the hair dye material comprises an oxidation base and the oxidation base is selected from para-phenylenediamines, bisphenylalkylenediamines, para-aminophenols, ortho-aminophenols and heterocyclic bases, and cosmetically acceptable salts of thereof.

85. The hair care kit of claim 83, wherein the hair dye material comprises a coupler and the coupler is selected from meta-phenylenediamines, meta-aminophenols, meta-diphenols, naphthalene couplers and heterocyclic couplers, and the cosmetically acceptable salts of thereof.

86. The hair care kit of claim 83, wherein the hair dye material comprises a direct dye and the direct dye is selected from nitro dyes of the benzene series, cationic direct dyes, azo direct dyes, and methine direct dyes.

87. The hair care kit of claim 82, wherein the oxidative agent is selected from peroxide enzymes and oxidase enzymes

88. The hair care kit of claim 87, wherein the oxidative agent is selected from peroxidases, oxidoreductases containing 2 electrons, and oxygenases containing 4 electrons.

89. The hair care kit of claim 88, wherein the oxidative agent is selected from uricases and laccases.

90. The hair care kit of claim 82, wherein the oxidative agent is hydrogen peroxide.

91. The hair care kit of claim 82, wherein the hair highlight material is selected from persalts.

92. The hair care kit of claim 91, wherein the hair highlight material comprises persulfate.

93. The hair care kit of claim 82, wherein the oxidative agent is further configured to be added to the hair light material to enhance the highlight power of the highlight material.

94. The hair care kit of claim 82, further comprising at least one highlight enhancing composition containing an oxidative agent chosen from hydrogen peroxide.

95. A hair care kit for a hair treatment process, the kit comprising:
at least one dye compartment comprising a hair dye material;
at least one highlight compartment comprising a hair highlight material; and
instructions for performing the hair treatment process, the instructions including instructions to apply to hair at least some of the hair dye material of said at least one dye compartment and at least some of the hair highlight material of said at least one highlight compartment,
wherein the instructions further comprise instructions for completing the hair treatment process in less than approximately 2 hours.

96. The hair care kit of claim 95, wherein the instructions for completing the hair treatment process include instructions for completing the process in less than approximately 1.5 hours.

97. The hair care kit of claim 96, wherein the instructions for completing the hair treatment process include instructions for completing the process in less than approximately 1 hour.

98. The hair care kit of claim 95, wherein the instructions for completing the hair treatment process include instructions for completing the process in about 1 hour.

99. A method for treating keratinous fibers, comprising:
applying a dye material to the keratinous fibers under conditions sufficient to dye the keratinous fibers; and
applying a highlight material to at least some of the keratinous fibers after applying the dye,
wherein the method comprises achieving multi-tonal highlighting of the keratinous fibers, and
wherein the method is completed in less than approximately 2 hours.

100. The method of claim 99, wherein the method is completed in less than approximately 1.5 hours.

101. The method of claim 100, wherein the method is completed in less than approximately 1 hour.

102. The method of claim 99, wherein the method is completed in about 1 hour.
